(19) United States
(12) Patent Application Publication
(10) Pub. No.: US 2009/0175875 A1
(43) Pub. Date: Jul. 9, 2009

(54) Molecules and Methods for Modulating Complement Component

(75) Inventors: Bijan ETEMAD-GILBERTSON, Jamaica Plain, MA (US); Braydon Charles Guild, Concord, MA (US); Mark Taylor Keating, Weston, MA (US); Yong-In Kim, Westborough, MA (US); Lloyd B. Klickstein, Newton, MA (US); Dmitri Mikhailov, Newton, MA (US); Mariusz Milik, Arlington, MA (US); Michael Roguska, Ashland, MA (US); Igor Splawski, Winchester, MA (US); Kehao Zhao, Newton, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/263,909

(22) Filed: Nov. 3, 2008

Related U.S. Application Data
(60) Provisional application No. 60/984,951, filed on Nov. 2, 2007.

(51) Int. Cl. A61K 39/395; C07K 16/00; A61P 27/02

(52) U.S. Cl. 424/139.1; 530/387.9; 530/387.3

(57) ABSTRACT

Compositions that bind to C3b epitopes and methods of using the compositions are described herein.

This application claims benefit under 35 U.S.C. § 119(a)-(d) or (f) or 365(b) of U.S. Application No. 60/984,951, filed Nov. 2, 2007, the contents of which are incorporated herein by reference in their entirety.

### TECHNICAL FIELD

This invention relates to antigen binding molecules, neo-epitopes bound by those molecules, and methods of using the molecules.

### BACKGROUND

Age related macular degeneration (AMD) is a progressive disease and a leading cause of vision loss and blindness in Americans aged 65 and older. AMD primarily affects the macula; a part of the retina responsible for high visual acuity needed to read or drive. The majority of AMD patients suffer from an early stage of the disease which is characterized by the presence of extracellular retinal deposits called drusen. Drusen are extracellular retinal deposits of cell debris, inflammatory mediators, and extracellular matrix components. The late stages of AMD manifest as a dry or wet form, both are associated with vision loss. Dry AMD, also known as geographic atrophy, appears on opthalmoscopic examination as clearly demarcated regions corresponding to local areas of retinal pigmented epithelium (RPE) loss. Wet AMD is associated with neo-vascularization of the choriod, causing a loss of integrity in Bruch's membrane and vessel growth in the retina, where they can often hemorrhage. This leakage causes permanent damage to retinal cells which die off and create blind spots in the central vision.

The innate human system is composed of the complement pathway. The complement pathway serves to defend against pyogenic bacterial infection bridging innate and adaptive immunity; and disposing of products of immune complexes and inflammatory injury. The complement is a system of more than 30 proteins involved in cascade reactions in plasma and cell surfaces. The complement system and its complement components are involved in various immune processes. For example, complement C5b-9 complex, also termed the terminal complex or the membrane attack complex (MAC), plays an important role in cell death by inducing membrane permeability damages.

Recent work has demonstrated that complement components C3 and C5 are principal constituents of drusen in patients with AMD. Mulling, R. F. et al. (2000) FASEB J 14, 835-46 Their presence as well as that of the membrane attack complex (MAC) C5b-9 and other acute phase reactant proteins in RPE cells overlying drusen has been speculated to be involved in the process that can trigger complement activation and formation of MAC. Johnson, L et al. (2001) Exp Eye Res 73, 887-896 Thus there is growing evidence that complement components are more than mere mediators of innate immunity.

Nutritional intervention has been prescribed to inhibit progression of dry AMD to wet AMD. At present the only FDA approved treatments for wet AMD include photodynamic therapy (PDT), an anti-VEGF aptamer, such as pegaptanib, and anti-VEGF antibodies, ranibizumab. These drugs or therapies are typically administered to patients who have already suffered substantial vision loss.

There remains a need to develop an effective treatment for AMD to replace or supplement current treatments. Particularly, there is a need for treatments which can provide early detection, prevention or restoration of vision loss.

### SUMMARY

The present invention relates to epitopes of complement component C3b, C3b binding molecules, and methods of making and using said molecules. The invention further provides molecules that bind to C3b (i.e., C3b binding molecules), particularly antibodies and portions thereof that bind human C3b epitopes and those modulate at least one complement protein or cellular activities mediated by the alternative and/or classical complement pathways.

Throughout the specification, reference to “complement pathways” or “complement” indicates either or both the alternative complement pathway or the classical complement pathway.

In one aspect, the complement component proteins whose level is to be modulated are anaphylotoxins, Factor H, Factor P, Factor B, C3 or C5 convertase; C3 cleavage products such as C3a, C3b, iC3b and C3d, C5 cleavage products C5a and C5b; MAC, and MAC-dependent production of complement by-products.

In another aspect, the binding molecules of the invention modulate enzymatic activity of a complement protein. In some methods, the enzymatic activity to be modulated is C3 and/or C5 convertase activity, conversion of C3 to C3a and C3b, conversion of C5 into C5a and C5b, and the formation of C5b-9.

In another aspect, the invention features a method of modulating the level of complement protein production in a subject. The method includes administering to the subject a C3b binding molecule that moderates one or more of the following biological activities: (a) inhibition of Factor P binding to C3 convertase; (b) inhibition of Factor B binding to C3b; (c) competitive or non-competitive inhibition of the proteolytic activity of the C3 or C5 convertase; (d) inhibiting binding of C3b to C3 convertase, thereby inhibiting formation of the C5 convertase; (e) inhibition of the formation of C3 cleavage products C3a, C3b, iC3b and C3d; (f) inhibition of the formation of C5 cleavage products C5a and C5b; (g) inhibition of MAC formation, and (h) inhibition of MAC-dependent production of complement by-products including C6, clusterin, haptoglobin, Ig kappa chain, Ig lambda chain, or Ig gamma chain. Some methods further comprise detecting the level of complement proteins in urine, blood plasma, serum, whole blood, or eye fluid from a subject.

Accordingly, in one aspect, the invention provides a C3b binding molecule including an antigen binding portion thereof that binds to a C3b neo-epitope, wherein the antigen binding portion binds to neo-epitopes selected from the group of amino acids listed in Table 1 below.

In another aspect, the C3b binding molecules have been altered in their affinity for an effector ligand. Anti-C3b antibodies of the invention preferably have mutations of leucines at positions 234 and 235 to alanines to abrogate FcRγ binding and attenuate effector functions.

Particularly, the invention provides an isolated C3b binding molecule comprising an antigen binding portion of an antibody that binds (e.g., specifically binds) to C3b neo-epitopes, wherein the antigen binding portion binds to a neo-epitope of human C3b within or overlapping one of the following C3b neo-epitopes: (a) GEDTVQSLTQG (amino acids 393-403, Seq ID No: 1); (b) DEDIIAEENIVSRSEF (amino acids 752-767, Seq ID No: 2); (c) IRMNKTVAVRT (amino acids 936-946, Seq ID No. 3); (d) SDQVPDTESET (amino acids 968-978, Seq ID No: 4); (e) VAQMTED (amino acids 987-993, Seq ID NO: 5), (f) FVKRAP (amino acids 1069-1074, Seq ID No: 6); (g) KDKNRWEDPGKQLYN (amino acids 1215-1229, Seq ID No: 7); (h) CTRYRGDQDATMS (amino acids 1389-1401, Seq ID No: 8); (i) GFAPDTDDLKQLANGV (amino acids 1410-1425, Seq ID No: 9).

In another aspect, the invention provides an isolated C3b binding molecule including an antigen binding portion of a binding molecule that binds (e.g., specifically binds) to a C3b neo-epitope linked to a second or third molecule. The second or third molecule may be free or attached in a complex, such as duplexes or triplexes. Such second or third molecule may be selected from the group consisting of C3b, C3bBb, C4b, C4b2a, Factor P, Factor H, Factor B or portions thereof.

In various aspects, the antigen binding portion specifically binds to a neo-epitope of human C3b within or overlapping one or more of the amino acids listed in Table 1 below. In various aspects, the binding molecule is an antibody or a molecule that functions in the manner of antibody which can bind a linear or non-linear epitope. In one aspect, where the C3b molecule binds to a non-linear epitope which includes one or more of the neo-epitopes of Table 1 herein, the neo-epitope should be conformationally arranged for the binding molecule to interact with one or more antigenic portions of the neo-epitope to substantially produce the biological function desired. Linear and non-linear neo-epitopes include at least one portion of each of the following linear epitopes: (a) amino acids 968-978 of SEQ ID NO: 4; and (b) amino acids 752-762 of SEQ ID NO: 2. In another example, the antigen binding portion binds to a non-linear neo-epitope including, or consisting of, at least one portion of each of the following linear neo-epitopes: (a) amino acids 936-946 of SEQ ID NO: 3; and (b) amino acids 1389-1401 of SEQ ID NO: 8. Any combination of non-linear neo-epitopes may be bound by such binding molecule to modulate at least one complement protein or cellular activities mediated by the complement pathway.

In other aspects, the C3b binding molecule is cross reactive with C3b of a non-human primate (e.g., a cynomolgus monkey, or a rhesus monkey). In various aspects, the antigen binding portion is cross reactive with a C3b of a rodent species (e.g., murine C3b, rat C3b, rabbit C3b).

In one aspect, the binding molecule of the present invention binds to C3b with a dissociation constant (K_{D}) equal to or less than 1 nM (e.g., 0.01 nM, 0.1 nM, 0.25 nM, 0.5 nM)

In another aspect, the C3b binding molecule binds to a C3b neo-epitope of a non-human primate (e.g., cynomolgus monkey or rhesus monkey) with a K_{D} within 5-10 fold of the K_{D} for binding to human C3b.

In one embodiment, the binding molecule of the present invention binds to mouse C3b neo-epitope with a K_{D} equal to or less than 5 nM or within 100-fold of the K_{D} for binding to human C3b.

In one aspect, the binding molecule is a chimeric (e.g., humanized) antibody or a human antibody.

In another aspect, the binding molecule is a monoclonal antibody or a polyclonal antibody.

The C3b binding molecule includes, for example, an Fab fragment, an Fab′ fragment, an F(ab′)₂, or an Fv fragment of the antibody.

In one aspect, the C3b binding molecule is a human antibody.

In one aspect, the C3b binding molecule includes a single chain Fv.

In one aspect, the C3b binding molecule includes a diabody (e.g., a single chain diabody, or a diabody having two polypeptide chains). In other aspects, the antigen binding portion of the antibody is derived from an antibody of one of the following isotypes: IgG1, IgG2, IgG3 or IgG4. In another aspect, the antigen binding portion of the antibody is derived from an antibody of an IgA or IgE isotype.

In another aspect, the invention provides compositions for eliciting antibodies that specifically bind to C3b neo-epitopes when the composition is administered to an animal. The compositions include, for example, one or more of the peptides listed in Table 1 herein; a peptide thereof with less than 5 amino acid changes; or a fragment thereof (e.g., fragments containing 2, 3, 4, 5, 6, 7, 8, 9, 10, 11, or 12 amino acids). The compositions can be modified to increase antigenicity, e.g., by coupling C3b neo-epitopes or fragments thereof to a carrier protein.

In another aspect, the invention features a method of decreasing MAC production in a cell. In one example, MAC production or inhibition may be measured by using standard CH50 and AH50 hemolytic assays, such as inhibition of hemolysis of red blood cells from chicken, rabbit, or humans. The assay method includes contacting red blood cells with a C3b binding molecule thereby inhibiting MAC formation on the red blood cell as further provided herein.

In some methods of the present invention, the binding molecules modulate cellular activity responsive to or mediated by the activated complement system. In some methods, the cellular activity to be modulated is cell lysis. Some methods further comprise detecting the cellular activity by, e.g., a hemolysis assay. In some methods, the cellular activity is detected in urine, blood plasma, serum, whole blood, or eye fluid from the subject.

The invention also provides a pharmaceutical composition that comprises one or more C3b binding molecules described herein. In one embodiment, the present invention provides a pharmaceutical composition comprising a C3b binding molecule (e.g., an antibody or an antigen binding fragment thereof) that binds to a human C3b epitope within or overlapping one of the C3b neo-epitopes selected from the group consisting of: (a) GEDTVQSLTQG (amino acids 393-403, Seq ID No: 1); (b) DEDIIAEENIVSRSEF (amino acids 752-767, Seq ID No: 2); (c) IRMNKTVAVRT (amino acids 936-946, Seq ID No. 3); (d) SDQVPDTESET (amino acids 968-978, Seq ID No: 4); (e) VAQMTED (amino acids 987-993, Seq ID NO: 5), (f) FVKRAP (amino acids 1069-1074, Seq ID No: 6); (g) KDKNRWEDPGKQLYN (amino acids 1215-1229, Seq ID No: 7); (h) CTRYRGDQDATMS (amino acids 1389-1401, Seq ID No: 8); (i) GFAPDTDDLKQLANGV (amino acids 1410-1425, Seq ID No: 9); and a pharmaceutically acceptable carrier.

In another embodiment, the present invention provides a pharmaceutical composition comprising a C3b binding molecule (e.g., an antibody or an antigen binding fragment thereof) that binds to a human C3b neo-epitope comprising at least one portion of each of the following linear epitopes: (a) amino acids 968-978 of SEQ ID NO: 4; and (b) amino acids 752-762 of SEQ ID NO: 2, and a pharmaceutically acceptable carrier. In one embodiment, the C3b binding molecule binds to a linear C3b neo-epitope. In another embodiment, the C3b binding molecule binds to a nonlinear C3b neo-epitope.

In another embodiment, the present invention provides a pharmaceutical composition comprising a C3b binding molecule (e.g., an antibody or an antigen binding fragment thereof) that binds to a human C3b non-linear neo-epitope comprising, or consisting of, at least one portion of each of the following linear epitopes: (a) amino acids 936-946 of SEQ ID NO: 3; and (b) amino acids 1389-1401 of SEQ ID NO: 8, and a pharmaceutically acceptable carrier.

In another aspect, the invention features a method of treating or preventing vision loss in a subject. As used herein, the term “treat” or “treatment” refers to any treatment of a disorder or disease in a subject, and includes, but is not limited to, preventing the disorder or disease from occurring in a subject which may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease, or relieving the condition caused by the disease or disorder, for example, stopping or ameliorating the symptoms of the disease or disorder. As used herein, the term “prevent” or “prevention,” in relation to a disease or disorder in a subject, means no disease or disorder development if none had occurred, or no further disorder or disease development if there had already been development of the disorder or disease. The method includes administering to the subject a pharmaceutical composition including a C3b binding molecule described herein in an amount effective to modulate an activity or level of at least one complement protein, or a cellular activity mediated by the complement pathway. In some methods, the subject has a condition or disorder associated with macular degeneration. In other methods, the subject is at risk of developing a disorder associated with macular degeneration. In some methods, the subject is free of complement-related diseases other than macular degeneration related disorders. In particular, the complement protein that is modulated is C3 convertase or C5 convertase enzymatic activity in a subject.

The amount that can be administered to a subject is be an amount effective to inhibit MAC, C5a production, or formation of C3 breakdown products (such as C3a, C3b, iC3b). In particular, the concentration of complement activation products (including but not limited to C3a and/or C5a) can be reduced in the subject's blood by at least 5%, 10%, 15%, 20%, 30%, 40%, 50%, 60% or 75% relative to baseline levels prior to administering the pharmaceutical composition.

Other diseases or disorders that can be treated with the methods of the present invention include, but not limited to, age-related macular disorder, North Carolina macular dystrophy, Sorsby's fundus dystrophy, Stargardt's disease, pattern dystrophy, Best disease, dominant drusen, and malattia leventinese retinal detachment, chorioretinal degenerations, retinal degenerations, photoreceptor degenerations, RPE degenerations, mucopolysaccharidoses, rod-cone dystrophies, cone-rod dystrophies, cone degenerations, glomerulonephritis, paroxysmal nocturnal hemoglobinuria (PNH), reducing the dysfunction of the immune and hemostatic systems associated with extracorporeal circulation, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, myocardial infarction, balloon angioplasty, post-pump syndrome in cardiopulmonary bypass or renal bypass, hemodialysis, renal ischemia, mesenteric artery reperfusion after acrotic reconstruction, infectious disease or sepsis, immune complex disorders and autoimmune diseases, rheumatoid arthritis, systemic lupus erythematosus (SLE), SLE nephritis, proliferative nephritis, hemolytic anemia, and myasthenia gravis. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, immune complex-associated inflammation, autoimmune heart disease, multiple sclerosis, inflammatory bowel disease, ischemia-reperfusion injuries, Barraquer-Simons Syndrome, hemodialysis, systemic lupus, lupus erythematosus, psoriasis, multiple sclerosis, transplantation, diseases of the central nervous system such as Alzheimer's disease and other neurodegenerative conditions, aHUS, bullous pemphigoid or MPGN II.

Pharmaceutical compositions of the invention may be administered via routes known in the art, for example, subcutaneously, intravenously, or intraocularly including intravitreally.

The details of one or more features of the invention are set forth in the accompanying drawings and the description below. Other features, objects, and advantages of the invention will be apparent from the description and drawing, and from the claims.

### DETAILED DESCRIPTION

In both the Classical and Alternative Complement Pathways, the inventors have discovered binding molecules which recognize and bind to C3b neo-epitopes, modulate the biological activity of C3 and/or C5 convertases and the generation of MAC. Such binding molecules can be used in preventing and/or treating diseases associated with abnormal activities of classical and/or alternative complement pathways, such as ocular disorders, conditions associated with macular degeneration, and the non-ocular disorders as described herein.

Accordingly, the present invention provides molecules that bind to C3b neo-epitopes, such as human antibodies and fragments thereof, which modulate complement proteins and/or cellular activities mediated by the complement pathway. Neo-epitopes of C3b and methods of making and using these neo-epitopes are also provided herein.

As used herein “neo-epitopes” or “neo-antigens” are used interchangeably and are antigenic portions of proteins that are present on C3b after proteolytic cleavage of C3. These neo-epitopes are not accessible on C3 which has not been cleaved.

The term “conditions or disorders associated with macular degeneration” refers to any of a number of conditions in which the retinal macula degenerates or becomes dysfunctional, e.g., as a consequence of decreased growth of cells of the macula, increased death or rearrangement of the cells of the macula (e.g., RPE cells), loss of normal biological function, or a combination of these events. Macular degeneration results in the loss of integrity of the histoarchitecture of the cells and/or extracellular matrix of the normal macula and/or the loss of function of the cells of the macula. Examples of macular degeneration-related disorder include AMD, North Carolina macular dystrophy, Sorsby's fundus dystrophy, Stargardt's disease, pattern dystrophy, Best disease, dominant drusen, and malattia leventinese (radial drusen). The term also encompasses extramacular changes that occur prior to, or following dysfunction and/or degeneration of the macula. Thus, the term “macular degeneration-related disorder” also broadly includes any condition which alters or damages the integrity or function of the macula (e.g., damage to the RPE or Bruch's membrane). For example, the term encompasses retinal detachment, chorioretinal degenerations, retinal degenerations, photoreceptor degenerations, RPE degenerations, mucopolysaccharidoses, rod-cone dystrophies, cone-rod dystrophies and cone degenerations.

The term “complement component”, “complement proteins” or “complement component proteins” refers to the molecules that are involved in activation of the complement system. The classical pathway components include, e.g., C1q, C1r, C1s, C4, C2, C3, C5, C6, C7, C8, C9, and C5b-9 complex (membrane attack complex: MAC). The alternative pathway components include, e.g., Factor B, Factor D, Properdin, H and I.

The terms “modulation” or “modulate” are used interchangeably herein to refer to both upregulation (i.e., activation or stimulation (e.g., by agonizing or potentiating) and downregulation (i.e., inhibition or suppression (e.g., by antagonizing, decreasing or inhibiting)) of an activity or a biological process (e.g., complement process). “Modulates” is intended to describe both the upregulation or downregulation of a process. A process which is upregulated by a certain stimulant may be inhibited by an antagonist to that stimulant. Conversely, a process that is downregulated by a certain modifying agent may be inhibited by an agonist to that modifying agent.

The terms “complement pathway associated molecules,” “complement pathway molecules,” and “complement pathway associated proteins” are used interchangeably and refer to the various molecules that play a role in complement activation and the downstream cellular activities mediated by, responsive to, or triggered by the activated complement system. They include initiators of complement pathways (i.e., molecules that directly or indirectly triggers the activation of complement system), molecules that are produced or play a role during complement activation (e.g., complement proteins/enzymes such as C3, C5, C5b-9, Factor B, Factor D, MASP-1, and MASP-2), complement receptors or inhibitors (e.g., clusterin, vitronectin, CR1, or CD59), and molecules regulated or triggered by the activated complement system (e.g., membrane attack complex-inhibitory factor, MACIF; see, e.g., Sugita et al., J Biochem, 106:589-92, 1989). Thus, in addition to complement proteins noted herein, complement pathway associated molecules also include, e.g., C3/C5 convertase regulators (RCA) such as complement receptor type 1 (also termed CR1 or CD35), complement receptor type 2 (also termed CR2 or CD21), membrane cofactor protein (MCP or CD46), and C4bBP; MAC regulators such as vitronectin, clusterin (also termed “SP40, 40”), CRP, CD59, and homologous restriction factor (HRF); immunoglobulin chains such as Ig kappa, Ig lambda, or Ig gamma); C1 inhibitor; and other proteins such as CR3, CR4 (CD11 b/18), and DAF (CD 55).

The term “cellular activities regulated by the complement pathway” include cell damage resulting from the C5b-9 attack complex, vascular permeability changes, contraction and migration of smooth muscle cells, T cell proliferation, immune adherence, aggregation of dendritic cells, monocytes, granulocyte and platelet, phagocytosis, migration and activation of neutrophils (PMN) and macrophages.

Further, activation of the complement pathways results in the increase of proinflammatory response contributed by the by-products within the complement pathway. Disorders associated with activation of the complement pathway include nephritis, asthma, reperfusion injury, hemodialysis, rheumatoid arthritis, systemic lupus, psoriasis, multiple sclerosis, transplantation, Alzheimer's disease, aHUS, MPGN II, or any other complement-mediated disease. Ddisorders associated with macular degeneration include AMD, North Carolina macular dystrophy, Sorsby's fundus dystrophy, Stargardt's disease, pattern dystrophy, Best disease, dominant drusen, and malattia leventinese (radial drusen), extramacular changes that occur prior to, or following dysfunction and/or degeneration of the macula, retinal detachment, chorioretinal degenerations retinal degenerations, photoreceptor degenerations, RPE degenerations, mucopolysaccharidoses, rod-cone dystrophies, cone-rod dystrophies and cone degenerations.

As used herein, the term “subject” includes any human or nonhuman animal.

The term “nonhuman animal” includes all nonhuman vertebrates, e.g., mammals and non-mammals, such as nonhuman primates, rodents, rabbits, sheep, dogs, cats, horses, cows, birds, amphibians, reptiles, etc.

The term “antibody” as used herein refers to an intact antibody or an antigen binding fragment (i.e., “antigen-binding portion”) or single chain (i.e., light or heavy chain) or mimetic thereof. An intact antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen binding portion” or “binding domain” of an antibody, as used herein, refers to one or more fragments of an intact antibody that retain the ability to specifically bind to a given antigen (e.g., C3b). Antigen binding functions of an antibody can be performed by fragments of an intact antibody. Examples of binding fragments encompassed within the term “antigen binding portion” of an antibody include a Fab fragment, a monovalent fragment consisting of the V_{L}, V_{H}, C_{L} and CH1 domains; an F(ab)₂ fragment, a bivalent fragment comprising two Fab fragments (generally one from a heavy chain and one from a light chain) linked by a disulfide bridge at the hinge region; an Fd fragment consisting of the V_{H} and CH1 domains; an Fv fragment consisting of the V_{L} and V_{H} domains of a single arm of an antibody; a single domain antibody (dAb) fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (V_{H}—CH1-V_{H}—CH1) which, together with complementary light chain polypeptides, form a pair of antigen binding regions (Zapata et al., 1995 Protein Eng. 8(10):1057-1062; and U.S. Pat. No. 5,641,870).

An “isolated C3b binding molecule”, as used herein, refers to a binding molecule that is substantially free of molecules having antigenic specificities for antigens other than C3b (e.g., an isolated antibody that specifically binds C3b is substantially free of antibodies that specifically bind antigens other than C3b such as C3) An isolated binding molecule that specifically binds C3b may, however, have cross-reactivity to other antigens, such as C3b molecules from other species. An isolated binding molecule is “purified” if it is substantially free of cellular material.

The term “monoclonal antibody composition” as used herein refers to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to an antibody displaying a single binding specificity that has variable regions in which both the framework and CDR regions are derived from human sequences. In one aspect, the human monoclonal antibody is produced by a hybridoma that includes a B cell obtained from a transgenic nonhuman animal (e.g., a transgenic mouse having a genome comprising a human heavy chain transgene and a light chain transgene) fused to an immortalized cell.

The term “recombinant human antibody”, as used herein, includes any human antibody that is prepared, expressed, created or isolated by recombinant means, such as an antibody isolated from an animal (e.g., a mouse) that is transgenic or transchromosomal for human immunoglobulin genes or a hybridoma prepared therefrom; an antibody isolated from a host cell transformed to express the human antibody, e.g., from a transfectoma; an antibody isolated from a recombinant, combinatorial human antibody library; and an antibody prepared, expressed, created or isolated by any other means that involve splicing of all or a portion of a human immunoglobulin gene sequences to another DNA sequence. Such recombinant human antibodies have variable regions in which the framework and CDR regions are derived from human germline immunoglobulin sequences. In certain aspects, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in a human.

As used herein, “isotype” refers to the antibody class (e.g., IgM, IgE, IgG such as IgG1 or IgG4) that is encoded by the heavy chain constant region gene.

The phrases “an antibody recognizing an antigen” and “an antibody specific for an antigen” are used interchangeably herein with the term “an antibody that binds specifically to an antigen.”

As used herein, the term “high affinity”, when referring to an IgG antibody, indicates that the antibody has a K_{D} of 10⁻⁹ M or less for a target antigen.

As used herein, a C3b binding molecule (e.g., an antibody or antigen binding portion thereof) that “specifically binds to C3b” is intended to refer to a C3b binding molecule that binds to C3b with a K_{D} of 1×10⁻⁷ M or less. Preferred binding molecules of the invention binds to a C3b neo-epitope with a K_{D} equal to or less than 1 nM (e.g., 0.01 nM, 0.1 nM, 0.25 nM, 0.5 nM).

A C3b binding molecule (e.g., an antibody) that cross-reacts with an antigen refers to a C3b binding molecule that binds that antigen with a K_{D} of 1×10⁻⁶ M or less. In a specific embodiment, a C3b binding molecule binds to a C3b neo-epitope of a non-human primate (e.g., cynomolgus monkey) with a K_{D} within 5-10 fold of the K_{D} to human. In another specific embodiment, a C3b binding molecule binds to a mouse C3b neo-epitope with a K_{D} equal to or within 100-fold to human.

A C3b binding molecule (e.g., an antibody) that does not cross-react with a given antigen refers to a C3b binding molecule that either does not bind detectably to the given antigen, or binds with a K_{D} of 1×10⁻⁵ M or greater. In certain aspects, such binding molecules that do not cross-react with the antigen exhibit essentially undetectable binding against these proteins in standard binding assays.

C3b Binding Molecules

Binding molecules of the invention bind to neo-epitopes of C3b having an amino acid sequence at least 90% identical to one or more of the following neo-epitopes:

TABLE 1

| | | | |
|---|---|---|---|
| | Amino Acid # | | |
| | (amino acid #1 is | | |
| C3b chain | initiation M | SEQ ID | |
| sequence | (methionine)) | Amino Acid Seq | NO |

| | | | |
|---|---|---|---|
| Beta | 393 | GEDTVQSLTQG | 1 |
| Alpha | 752 | DEDIIAEENIVSRSEF | 2 |
| Alpha | 936 | IRMNKTVAVRT | 3 |
| Alpha | 968 | SDQVPDTESET | 4 |
| Alpha | 987 | VAQMTED | 5 |
| Alpha | 1069 | FVKRAP | 6 |
| Alpha | 1215 | KDKNRWEDPGKQLYN | 7 |
| Alpha | 1388 | CTRYRGDQDATMS | 8 |
| Alpha | 1410 | GFAPDTDDLKQLANGV | 9 |
| Beta | 178 | DSLSSQNQLGVL | 10 |
| Beta | 292 | PIEDGSGEVVLSRK | 11 |
| Beta | 310 | GVQNPRAEDLVG | 12 |
| Beta | 380 | DGSPAYR | 13 |
| Beta | 392 | QGEDTVQSL | 14 |
| Beta | 428 | KQELSEAE | 15 |
| Beta | 507 | VREPGQDLVVLP | 16 |
| Beta | 564 | VVKSGQSEDRQPVPG | 17 |
| Alpha | 775 | VEDLKEPPKN | 18 |
| Alpha | 852 | YNYRQNQELKVR | 19 |
| Alpha | 876 | ATTKRRHQQT | 20 |
| Alpha | 919 | HFISDGVRKSLK | 21 |
| Alpha | 968 | SDQVPDTESET | 22 |
| Alpha | 1006 | TPSGOGEQN | 23 |
| Alpha | 1047 | ELIKKGYT | 24 |
| Alpha | 1110 | EKQKPDGVFQED | 25 |
| Alpha | 1133 | LRNNNEKDM | 26 |
| Alpha | 1212 | TTAKDKNRWEDPGKQ | 27 |
| Alpha | 1388 | CTRYRGDQDATMS | 28 |
| Alpha | 1410 | GFAPDTDDLKQLANGV | 29 |
| Alpha | 1453 | HSEDDCLAFK | 30 |
| Alpha | 1571 | SGSDEVQVGQQR | 31 |
| Alpha | 1607 | LSSDFWGEKPNL | 32 |
| Alpha | 1634 | EDECQDEENQKQCQD | 33 |
| Beta | 94 | NREFKSEKG | 34 |
| Beta | 404 | QNL | 35 |
| Alpha | 1368 | ETEKRPQDA | 36 |
| Alpha | 1517 | SD | 37 |

The amino acids of Table 1 are numbered according to guidelines illustrated in the CO3_HUMAN entry in the SwissProt database (www.expasy.org).

A C3b binding molecule may bind specifically to linear or non-linear epitopes, including neo-epitopes selected from Table 1. Included in this invention are binding molecules that bind to non-linear epitopes that modulate C3b bioactivity.

C3b binding molecules include, for example, antibodies that bind to C3b neo-epitopes (in either free or complexed form), and polypeptides that include antigen binding portions of such antibodies. C3b binding molecules also include molecules in which the binding portion is not derived from an antibody, e.g., C3b binding molecules derived from polypeptides that have an immunoglobulin-like fold, and in which the antigen binding portion is engineered to bind C3b neo-epitopes through randomization, selection, and affinity maturation. Preferred C3b binding molecules include antibodies, fragments thereof or artificial constructs comprising antibodies or fragments thereof or artificial constructs designed to mimic the binding of antibodies or fragments thereof.

The invention also features C3b binding molecules which are not antibodies. Such C3b binding molecules include a C3b binding domain that has an amino acid sequence at least 60%, 65%, 75%, 80%, 85%, or 90% identical to an amino acid derived from an immunoglobulin-like (Ig-like) fold of a non-antibody polypeptide, such as one of the following: tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, α-glucuronidase, transglutaminase, T-cell antigen receptor, superoxide dismutase, tissue factor domain, cytochrome F, green fluorescent protein, GroEL, or thaumatin. In general, the amino acid sequence of the C3b binding domain is altered, relative to the amino acid sequence of the immunoglobulin-like fold, such that the C3b binding domain specifically binds to a C3b neo-epitope (i.e., wherein the immunoglobulin-like fold does not specifically bind to the C3).

The amino acid sequence of the C3b binding domain is at least 60% identical (e.g., at least 65%, 75%, 80%, 85%, or 90% identical) to an amino acid sequence of an immunoglobulin-like fold of a fibronectin, a cytokine receptor, or a cadherin.

A C3b binding molecule that specifically bind and modulate one or more of a number of bioactivities of C3b, Thus, the present invention features a C3b binding molecule that inhibits C3b binding of properdin, factor H, factor B, factor I, membrane cofactors, and/or complexes thereof. The C3b binding molecule also inhibits C3b formation of MAC by at least 5%, 10%, 15%, 25%, or 50%, relative to a control (e.g., relative to binding in the absence of a C3b binding molecule).

The C3b binding molecule of the present invention inhibits C3b binding to C3 convertase (e.g. the bimolecular complex C3bBb) to block formation of C5 convertase (e.g., C3bBbC3b, the trimolecular complex) in the alternative pathway. In another aspect, the C3b binding molecule inhibits C3b binding to the C3 convertase (e.g., the bimolecular complex C4bC2a) to block formation of C5 convertase (e.g., the trimolecular complex C3bC4bC2a) in the classical pathway. These biological activities are produced by competitive binding mechanism within the feedback loop involving C3 protein cleavage. Accordingly, the C3b binding molecule inhibits C3 cleavage by at least 5%, 10%, 15%, 25%, or 50%, relative to a control (e.g., relative to activity in the absence of the C3b binding molecule).

A C3b binding molecule that inhibits or modulates one or more of C3b bioactivities (e.g., biochemical, cellular, physiological or other biological activities as a result of complement pathway activation), as determined according to methodologies known to the art and described herein, will be understood to produce a statistically significant decrease in the particular functional property relative to that seen in the absence of the C3b binding molecule (e.g., when a control molecule of irrelevant specificity is present). A C3b binding molecule that modulates C3b bioactivity effects such a statistically significant decrease by at least 5% of the measured parameter. In certain aspects, a C3b binding molecule may produce a decrease in the selected functional property of at least 10%, 20%, 30%, or 50% compared to control.

Standard assays to evaluate the ability of molecules to bind to C3b of various species, and particular epitopes of C3b, are known in the art, including, for example, ELISAs and Western blots. Determination of whether a C3b binding molecule binds to a specific epitope of C3b can employ a peptide epitope competition assay. For example, a C3b binding molecule is incubated with a peptide corresponding to a C3b epitope of interest at saturating concentrations of peptide. The preincubated C3b binding molecule is tested for binding to immobilized C3b, e.g., by Biacore analysis. Inhibition of C3b binding by preincubation with the peptide indicates that the C3b binding molecule binds to the peptide epitope (see, e.g., U.S. Pat. Pub. 20070072797). Binding kinetics also can be assessed by standard assays known in the art, such as by Biacore analysis. Assays to evaluate the effects of C3b binding molecules on functional properties of C3b are described in further detail below.

C3b inhibition may be determined by measuring, for example; (a) the ability of patient serum to block red blood cell hemolysis in an in vitro assay; (b) serum C3a or C5a levels; (c) soluble MAC levels in plasma, tissue, and or other biologic components, such as the ocular material or components. A decrease in C5a, C3a or C5b-9 levels in the presence of a C3b binding molecule indicates that the C3b binding molecule inhibits C3b and/or its bioactivity.

Various biological samples from a subject can be used for the detection, e.g., samples obtained from any organ, tissue, or cells, as well as blood, urine, or other bodily fluids (e.g., eye fluid). For some diagnostic methods, a preferred sample is eye fluid. For some other methods, a preferred tissue sample is whole blood and products derived therefrom, such as plasma and serum. Blood samples can be obtained from blood-spot taken from, for example, a Guthrie card. Other sources of tissue samples are skin, hair, urine, saliva, semen, feces, sweat, milk, amniotic fluid, liver, heart, muscle, kidney and other body organs. Others sources of tissue are cell lines propagated from primary cells from a subject. Tissue samples are typically lysed to release the protein and/or nucleic acid content of cells within the samples. The protein fraction from such crude lysates can then be subject to partial or complete purification before analysis

Other subjects who are amenable to treatment with the C3b binding molecules of the invention include individuals free of known complement related diseases other than macular degeneration-related disorders. Complement related diseases or disorders have been described in the art, e.g., in U.S. Pat. No. 6,169,068. Examples of known complement related diseases include: neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, myocardial infarction, balloon angioplasty, post-pump syndrome in cardiopulmonary bypass or renal bypass, hemodialysis, renal ischemia, mesenteric artery reperfusion after acrotic reconstruction, infectious disease or sepsis, immune complex disorders and autoimmune diseases, rheumatoid arthritis, systemic lupus erythematosus (SLE), SLE nephritis, proliferative nephritis, hemolytic anemia, and myasthenia gravis. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's syndrome, pulmonary vasculitis, and immune complex-associated inflammation.

Subjects to be treated with therapeutic agents of the present invention can also be administered other therapeutic agents with know methods of treating conditions associated with macular degeneration, such as antibiotic treatments as described in U.S. Pat. No. 6,218,368. In other treatments, immunosuppressive agents such as cyclosporine, are agents capable of suppressing immune responses. These agents include cytotoxic drugs, corticosteroids, nonsteroidal anti-inflammatory drugs (NSAIDs), specific T-lymphocyte immunosuppressants, and antibodies or fragments thereof (see Physicians' Desk Reference, 53rd edition, Medical Economics Company Inc., Montvale, N.J. (1999). Immunosuppressive treatment is typically continued at intervals for a period of a week, a month, three months, six months or a year. In some patients, treatment is administered for up to the rest of a patient's life.

Antibodies

Anti-C3b antibodies described herein include human monoclonal antibodies. In some aspects, antigen binding portions of antibodies that bind to C3b, (e.g., V_{H} and V_{L} chains) are “mixed and matched” to create other anti-C3b binding molecules. The binding of such “mixed and matched” antibodies can be tested using the aforementioned binding assays (e.g., ELISAs). When selecting a V_{H} to mix and match with a particular V_{L} sequence, typically one selects a V_{H} that is structurally similar to the V_{H} it replaces in the pairing with that V_{L}. Likewise a full length heavy chain sequence from a particular full length heavy chain/full length light chain pairing is generally replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Identifying structural similarity in this context is a process well known in the art.

In other aspects, the invention provides antibodies that comprise the heavy chain and light chain CDR1s, CDR2s and CDR3s of one or more C3b-binding antibodies, in various combinations. Given that each of these antibodies can bind to C3b and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and matched). C3b binding of such “mixed and matched” antibodies can be tested using the binding assays described herein (e.g., ELISAs). When V_{H} CDR sequences are mixed and matched, the CDR1, CDR2 and/or CDR3 sequence from a particular V_{H} sequence should be replaced with a structurally similar CDR sequencers). Likewise, when V_{L} CDR sequences are mixed and matched, the CDR1, CDR2 and/or CDR3 sequence from a particular V_{L} sequence should be replaced with a structurally similar CDR sequencers). Identifying structural similarity in this context is a process well known in the art.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes as the source of the sequences. In one such system, a human antibody is raised in a transgenic mouse carrying human immunoglobulin genes. The transgenic mouse is immunized with the antigen of interest (e.g., a neo-epitope of C3b described herein). Alternatively, a human antibody is identified by providing a human immunoglobulin gene library displayed on phage and screening the library with the antigen of interest (e.g., C3b or a C3b neo-epitope described herein).

A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germine immunoglobulin sequence may contain amino acid differences as compared to the germline-encoded sequence, due to, for example, naturally occurring somatic mutations or artificial site-directed mutations. However, a selected human antibody typically has an amino acid sequence at least 90% identical to an amino acid sequence encoded by a human germline immunoglobulin gene and contains amino acid residues that identify the human antibody as being human when compared to the germine immunoglobulin amino acid sequences of other species (e.g., murine germine sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

The percent identity between two sequences is a function of the number of identity positions shared by the sequences (i.e., % identity=# of identity positions/total # of positions×100), taking into account the number of gaps, and the length of each gap, that need to be introduced for optimal alignment of the two sequences. The comparison of sequences and determination of percent identity between two sequences is determined using the algorithm of E. Meyers and W. Miller (1988 Comput. Appl. Biosci., 4:11-17) which has been incorporated into the ALIGN program (version 2.0), using a PAM120 weight residue table, a gap length penalty of 12 and a gap penalty of 4.

Typically, a V_{H} or V_{L} of a human antibody derived from a particular human germline sequence will display no more than 10 amino acid differences from the amino acid sequence encoded by the human germline immunoglobulin gene. In certain cases, the V_{H} or V_{L} of the human antibody may display no more than 5, or even no more than 4, 3, 2, or 1 amino acid difference from the amino acid sequence encoded by the germline immunoglobulin gene.

Camelid Antibodies

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for C3b. In certain aspects herein, the camelid antibody or nanobody is naturally produced in the camelid animal, i.e., is produced by the camelid following immunization with C3b or a peptide fragment thereof, using techniques described herein for other antibodies. Alternatively, an anti-C3b camelid nanobody is engineered, i.e., produced by selection, for example from a library of phage displaying appropriately mutagenized camelid nanobody proteins using panning procedures with C3b or a C3b neo-epitope described herein as a target. Engineered nanobodies can further be customized by genetic engineering to increase the half-life in a recipient subject from 45 minutes to two weeks.

Diabodies

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fc to generate a “di-diabody” (see Lu et al., 2004J. Biol. Chem., 279(4):2856-65).

Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest (e.g., C3b antigen). For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another aspect, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other aspects, the Fc region is altered by replacing at least one amino acid residue with a different amino acid residue to alter the effector functions of the antibody. The effector ligand to which affinity is altered can be, for example, an Fc receptor or the C1 component of complement. For example, one or more amino acids can be replaced with a different amino acid residue such that the antibody has an altered affinity for an effector ligand but retains the antigen-binding ability of the parent antibody. Exemplary amino acid mutations occur at positions selected from 234, 235, 236, 237, 252, 254, 256, 297, 309, 311, 315, 318, 320, 322, 433 and/or 434. C3b binding molecules of the invention specifically encompass consensus Fc antibody domains prepared and used according to the teachings of this invention. Preferred anti-C3b antibodies include Fc mutations at positions selected from 234 and/or 235. This approach is described in detail in U.S. Pat. Nos. 5,624,821 and 5,648,260, both by Winter et al.

In another aspect, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another aspect, one or more amino acid residues are altered to thereby alter the ability of the antibody to fix complement. This approach is described further in WO 94/29351 by Bodmer et al.

In yet another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In still another aspect, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain aspects, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of a C3b binding polypeptide of the invention by the introduction of a normatural amino acid. Certain normatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the normatural amino acid of choice. Particular normatural amino acids that are beneficial for purpose of conjugating moieties to the polypeptides of the invention include those with acetylene and azido side chains. The polypeptides containing these novel amino acids can then be pegylated at these chosen sites in the protein.

Methods of Engineering Antibodies

As discussed above, anti-C3b antibodies can be used to create new anti-C3b antibodies by modifying full length heavy chain and/or light chain sequences, V_{H} and/or V_{L} sequences, or the constant region(s) attached thereto. For example, one or more CDR regions of the antibodies can be combined recombinantly with known framework regions and/or other CDRs to create new, recombinantly-engineered, anti-C3b antibodies. Other types of modifications include those described in the previous section. The starting material for the engineering method is one or more of the V_{H} and/or V_{L} sequences, or one or more CDR regions thereof. To create the engineered antibody, it is not necessary to actually prepare (i.e., express as a protein) an antibody having one or more of the V_{H} and/or V_{L} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the functional properties of the anti-C3b antibody from which it is derived, which functional properties include, but are not limited to, specifically binding to C3b, inhibiting formation of C3b complexes, inhibiting C3 convertase activation, inhibiting C5 convertase activation, inhibiting formation of MAC. The functional properties of the altered antibodies can be assessed using standard assays available in the art and/or described herein (e.g., ELISAs).

In certain aspects of the methods of engineering antibodies of the invention, mutations can be introduced randomly or selectively along all or part of an anti-C3b antibody coding sequence and the resulting modified anti-C3b antibodies can be screened for binding activity and/or other functional properties (e.g., inhibiting C3 or C5 convertase activity, inhibiting MAC formation, modulating complement pathway dysregulation) as described herein. Mutational methods have been described in the art. For example, PCT Pub. WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

A nucleotide sequence is said to be “optimized” if it has been altered to encode an amino acid sequence using codons that are preferred in the production cell or organism, generally a eukaryotic cell, for example, a cell of a yeast such as Pichia, an insect cell, a mammalian cell such as Chinese Hamster Ovary cell (CHO) or a human cell. The optimized nucleotide sequence is engineered to encode an amino acid sequence identical or nearly identical to the amino acid sequence encoded by the original starting nucleotide sequence, which is also known as the “parental” sequence.

Non-Antibody C3b Binding Molecules

The invention further provides C3b binding molecules that exhibit functional properties of antibodies but derive their framework and antigen binding portions from other polypeptides (e.g., polypeptides other than those encoded by antibody genes or generated by the recombination of antibody genes in vivo). The antigen binding domains (e.g., C3b binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1,1-set immunoglobulin domain of myosin-binding protein C, I-set immunoglobulin domain of myosin-binding protein H, I-set immunoglobulin domain of telokin NCAM, twitchin, neuroglian, growth hormone receptor, erythropoietin receptor, prolactin receptor, interferon-gamma receptor, □-galactosidase/glucuronidase, □-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., C3b) and for other functions (e.g., inhibition of biological activity of C3b). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

Nucleic Acid Molecules Encoding Antibodies of the Invention

Another aspect of the invention pertains to nucleic acid molecules that encode the C3b binding molecules of the invention. The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an aspect, the nucleic acid is a cDNA molecule. The nucleic acid may be present in a vector such as a phage display vector, or in a recombinant plasmid vector.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or a lambda constant region.

To create an scFv gene, the V_{H}— and V_{L}-encoding DNA fragments are operatively linked to another fragment encoding a flexible linker, e.g., encoding the amino acid sequence (Gly4-Ser)₃, such that the V_{H} and V_{L} sequences can be expressed as a contiguous single-chain protein, with the V_{L} and V_{H} regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554).

Monoclonal Antibody Generation

Monoclonal antibodies (mAbs) can be produced by a variety of techniques, including conventional monoclonal antibody methodology e.g., the standard somatic cell hybridization technique of Kohler and Milstein (1975 Nature, 256:495), or using library display methods, such as phage display.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539, and U.S. Pat. Nos. 5,530,101; 5,585,089; 5,693,762 and 6,180,370.

In a certain aspect, the antibodies of the invention are human monoclonal antibodies. Such human monoclonal antibodies directed against C3b epitopes can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see, e.g., Lonberg et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Pub. Nos. WO 92103918, WO 93/12227, WO 94/25585, WO 97113852, WO 98/24884 and WO 99/45962, all to Lonberg and Kay; and PCT Pub. No. WO 01/14424 to Korman et al.

In another aspect, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in WO 02/43478.

Still further, alternative transgenic animal systems expressing human immunoglobulin genes are available in the art and can be used to raise anti-C3b antibodies of the invention. For example, an alternative transgenic system referred to as the Xenomouse® (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-C3b antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise anti-C3b antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Libraries can be screened for binding to full length C3b antigen or to a particular C3b neo-epitope.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Generation of Human Monoclonal Antibodies in Human Ig Mice

Purified recombinant human C3b expressed in prokaryotic cells (e.g., E. coli) or eukaryotic cells (e.g., mammalian cells, e.g., HEK293 cells) can be used as the antigen. The protein can be conjugated to a carrier, such as keyhole limpet hemocyanin (KLH).

Fully human monoclonal antibodies to C3b neo-epitopes are prepared using HCo7, HCo12 and HCo17 strains of HuMab transgenic mice and the KM strain of transgenic transchromosomic mice, each of which express human antibody genes. In each of these mouse strains, the endogenous mouse kappa light chain gene can be homozygously disrupted as described in Chen et al., 1993 EMBO J. 12:811-820 and the endogenous mouse heavy chain gene can be homozygously disrupted as described in Example 1 of WO 01109187. Each of these mouse strains carries a human kappa light chain transgene, KCo5, as described in Fishwild et al., 1996 Nature Biotechnology 14:845-851. The HCo7 strain carries the HCo7 human heavy chain transgene as described in U.S. Pat. Nos. 5,545,806; 5,625,825; and 5,545,807. The HCo12 strain carries the HCo12 human heavy chain transgene as described in Example 2 of WO 01/09187. The HCo17 stain carries the HCo17 human heavy chain transgene. The KNM strain contains the SC20 transchromosome as described in WO 02/43478.

To generate fully human monoclonal antibodies to C3b neo-epitopes, HuMab mice and KM mice are immunized with purified recombinant C3b, a C3b fragment, or a conjugate thereof (e.g., C3b-KLH) as antigen. General immunization schemes for HuMab mice are described in Lonberg, N. et al., 1994 Nature 368(6474): 856-859; Fishwild, D. et al., 1996 Nature Biotechnology 14:845-851 and WO 98/24884. The mice are 6-16 weeks of age upon the first infusion of antigen. A purified recombinant preparation (5-50 μg) of the antigen is used to immunize the HuMab mice and KM mice in the peritoneal cavity, subcutaneously (Sc) or by footpad injection.

Transgenic mice are immunized twice with antigen in complete Freund's adjuvant or Ribi adjuvant either in the peritoneal cavity (IP), subcutaneously (Sc) or by footpad (FP), followed by 3-21 days IP, Sc or FP immunization (up to a total of 11 immunizations) with the antigen in incomplete Freund's or Ribi adjuvant. The immune response is monitored by retroorbital bleeds. The plasma is screened by ELISA, and mice with sufficient titers of anti-C3b human immunoglobulin are used for fusions. Mice are boosted intravenously with antigen 3 and 2 days before sacrifice and removal of the spleen. Typically, 10-35 fusions for each antigen are performed. Several dozen mice are immunized for each antigen. A total of 82 mice of the HCo7, HCo12, HCo17 and KM mice strains are immunized with C3b antigens.

To select HuMab or KM mice producing antibodies that bound C3b neo-epitopes, sera from immunized mice can be tested by ELISA as described by Fishwild, D. et al., 1996. Briefly, microtiter plates are coated with purified recombinant C3b at 1-2 μg/ml in PBS, 50 μl/wells incubated 4° C. overnight then blocked with 200 μl/well of 5% chicken serum in PBS/Tween (0.05%). Dilutions of plasma from C3b-immunized mice are added to each well and incubated for 1-2 hours at ambient temperature. The plates are washed with PBS/Tween and then incubated with a goat-anti-human IgG Fc polyclonal antibody conjugated with horseradish peroxidase (HRP) for 1 hour at room temperature. After washing, the plates are developed with ABTS substrate (Sigma, A-1888, 0.22 mg/ml) and analyzed by spectrophotometer at OD 415-495. Splenocytes of mice that developed the highest titers of anti-C3b antibodies are used for fusions. Fusions are performed and hybridoma supernatants are tested for anti-C3b activity by ELISA.

The mouse splenocytes, isolated from the HuMab mice and KM mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of SP2/0 nonsecreting mouse myeloma cells (ATCC, CRL 1581) with 50% PEG (Sigma). Cells are plated at approximately 1×10⁵/well in flat bottom microtiter plates, followed by about two weeks of incubation in selective medium containing 10% fetal bovine serum, 10% P388D 1 (ATCC, CRL TIB-63) conditioned medium, 3-5% Origen® (IGEN) in DMEM (Mediatech, CRL 10013, with high glucose, L-glutamine and sodium pyruvate) plus 5 mM HEPES, 0.055 mM 2-mercaptoethanol, 50 μg/ml gentamycin and 1×HAT (Sigma, CRL P-7185). After 1-2 weeks, cells are cultured in medium in which the HAT is replaced with HT. Individual wells are then screened by ELISA for human anti-C3b monoclonal IgG antibodies. Once extensive hybridoma growth occurred, medium is monitored usually after 10-14 days. The antibody secreting hybridomas are replated, screened again and, if still positive for human IgG, anti-C3b monoclonal antibodies are subcloned at least twice by limiting dilution. The stable subclones are then cultured in vitro to generate small amounts of antibody in tissue culture medium for further characterization.

Generation of Hybridomas Producing Human Monoclonal Antibodies

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% Origen® (IGEN), 4 mM L-glutamine, 1 mM sodium pyruvate, 5 mM HEPES, 0:055 mM 2-mercaptoethanol, 50 units/ml penicillin, 50 μg/ml streptomycin, 50 μg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using an extinction coefficient of 1.43. The monoclonal antibodies can be aliquoted and stored at −80° C.

Generation of Transfectomas Producing Monoclonal Antibodies

Antibodies of the invention also can be produced in a host cell transfectoma using, for example, a combination of recombinant DNA techniques and gene transfection methods as is well known in the art (e.g., Morrison, 1985 Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. 1990 Methods in Enzymology 185, Academic Press, San Diego, Calif.). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988 Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood, 1985 Immunology Today 6:12-13).

Mammalian host cells for expressing the recombinant antibodies of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in Kaufman and Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

Bispecific Molecules

In another aspect, the present invention features bispecific molecules comprising a C3b binding molecule (e.g., an anti-C3b antibody, or a fragment thereof), of the invention. A C3b binding molecule of the invention can be derivatized or linked to another functional molecule, e.g., another peptide or protein (e.g., another antibody or ligand for a receptor) to generate a bispecific molecule that binds to at least two different binding sites or target molecules. The C3b binding molecule of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for C3b neo-epitopes and a second binding specificity for a second target epitope such as Factor B, Factor D, Properdin, Factor H, Factor I or complement proteins/enzymes involved in generation of MAC, such as C5, C6, C7, C8, and C9.

In one aspect, the bispecific molecules of the invention comprise as a binding specificity at least one antibody, or an antibody fragment thereof including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

The bispecific molecules of the present invention can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al., 1984 J. Exp. Med. 160:1686; Liu et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly aspect, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

Screening and Assays

### Complement Activation Assays

The functional characteristics of C3b binding molecules can be tested in vitro and in vivo. For example, binding molecules can be tested for the ability to inhibit interaction of C3b and complement proteins such as properdin, factor H, factor B, factor I, membrane cofactors, and/or complexes thereof. Further binding molecules can be tested for its ability to inhibit C3 and/or C5 convertase activity according to Wiesmann, C, et al. (2006). Nature 444, 217-220.

Various methods can be used to measure activities of complement pathway molecules and activation of the complement system (see, e.g., U.S. Pat. No. 6,087,120; and Newell et al., J Lab Clin Med, 100:437-44, 1982). For example, the complement activity can be monitored by (i) measurement of inhibition of complement-mediated lysis of red blood cells (hemolysis); (ii) measurement of ability to inhibit cleavage of C3 or C5; and (iii) inhibition of classical and/or alternative pathway mediated hemolysis.

The two most commonly used techniques are hemolytic assays (see, e.g., Baatrup et al., Ann Rheum Dis, 51:892-7, 1992) and immunological assays (see, e.g., Auda et al., Rheumatol Int, 10:185-9, 1990). The hemolytic techniques measure the functional capacity of the entire sequence-either the classical or alternative pathway. Immunological techniques measure the protein concentration of a specific complement component or split product. Other assays that can be employed to detect complement activation or measure activities of complement components in the methods of the present invention include, e.g., T cell proliferation assay (Chain et al., J Immunol Methods, 99:221-8, 1987), and delayed type hypersensitivity (DTH) assay (Forstrom et al., 1983, Nature 303:627-629; Halliday et al., 1982, in Assessment of Immune Status by the Leukocyte Adherence Inhibition Test, Academic, New York pp. 1-26; Koppi et al., 1982, Cell. Immunol. 66:394-406; and U.S. Pat. No. 5,843,449).

In hemolytic techniques, all of the appropriate complement components must be present and functional (depending on the pathway that being measured the required components may vary). Therefore hemolytic techniques can screen both functional integrity and deficiencies of the complement system (see, e.g., Dijk et al., J Immunol Methods 36: 29-39, 1980; Minh et al., Clin Lab Haematol. 5:23-34 1983; and Tanaka et al., J Immunol 86: 161-170, 1986). For example, to measure the functional capacity of the classical pathway, sheep red blood cells (red blood cells from other species can be used as well, e.g., chicken red blood cells can be used) coated with hemolysin (rabbit IgG to sheep red blood cells) are used as target cells (sensitized cells). These Ag-Ab complexes activate the classical pathway and result in lysis of the target cells when the components are functional and present in adequate concentration. To determine the functional capacity of the alternative pathway, rabbit red blood cells are used as the target cell (see, e.g., U.S. Pat. No. 6,087,120).

The hemolytic complement measurement is applicable to detect deficiencies and functional disorders of complement proteins, e.g., in the blood of a subject, since it is based on the function of complement to induce cell lysis, which requires a complete range of functional complement proteins. The so-called CH50 method, which determines classical pathway activation, and the AP50 method for the alternative pathway have been extended by using specific isolated complement proteins instead of whole serum, while the highly diluted test sample contains the unknown concentration of the limiting complement component. By this method a more detailed measurement of the complement system can be performed, indicating which component is deficient.

Immunologic techniques employ polyclonal or monoclonal antibodies against the different epitopes of the various complement components (e.g., C3, C4 an C5) to detect, e.g., the split products of complement components (see, e.g., Hugli et al., Immunoassays Clinical Laboratory Techniques 443-460, 1980; Gorski et al., J Immunol Meth 47: 61-73, 1981; Linder et al., J Immunol Meth 47: 49-59, 1981; and Burger et al., J Immunol 141: 553-558, 1988). Binding of the antibody with the split product in competition with a known concentration of labeled split product could then be measured. Various assays such as radio-immunoassays, ELISA's, and radial diffusion assays are available to detect complement split products.

The immunologic techniques provide a high sensitivity to detect complement activation, since they allow measurement of split-product formation in blood from a test subject and control subjects with or without macular degeneration-related disorders. Accordingly, in some methods of the present invention, diagnosis of a disorder associated with macular degeneration is obtained by measurement of abnormal complement activation through quantification of the soluble split products of complement components (e.g., C3a, C4a, C5a, and the C5b-9 terminal complex) in blood plasma from a test subjects. The measurements can be performed as described, e.g., in Chenoweth et al., N Engl J Med 304: 497-502, 1981; and Bhakdi et al., Biochim Biophys Acta 737: 343-372, 1983. Preferably, only the complement activation formed in vivo is measured. This can be accomplished by collecting a biological sample from the subject (e.g., serum) in medium containing inhibitors of the complement system, and subsequently measuring complement activation (e.g., quantification of the split products) in the sample.

In the clinical diagnosis or monitoring of patients with disorders associated with macular degeneration, the detection of complement proteins in comparison to the levels in a corresponding biological sample from a normal subject is indicative of a patient with disorders associated with macular degeneration

The in vivo diagnostic or imaging is described in US2006/0067935. Briefly, these methods generally comprise administering or introducing to a patient a diagnostically effective amount of a C3b binding molecule that is operatively attached to a marker or label that is detectable by non-invasive methods. The antibody-marker conjugate is allowed sufficient time to localize and bind to complement proteins within the eye. The patient is then exposed to a detection device to identify the detectable marker, thus forming an image of the location of the C3b binding molecules in the eye of a patient. The presence of C3b binding molecules or complexes thereof is detected by determining whether an antibody-marker binds to a component of the eye. Detection of or an increased level in selected complement proteins or a combination thereof in comparison to a normal individual without AMD disease is indicative of a predisposition for and/or on set of disorders associated with macular degeneration. These aspects of the invention are also preferred for use in eye imaging methods and combined angiogenic diagnostic and treatment methods.

### Animal Models

Animal models suitable for testing C3b modulation by C3b binding molecules have been described in US2006/0067935. Animal models of AMD have been developed in mice, which develop pathological features seen in the human condition. Ambati, J et al, (2003) Nat Med 9, 1390-1397.

Toxicity and therapeutic efficacy of C3b binding molecules can be determined by standard pharmaceutical procedures in these experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The data obtained from the animal studies can be used in formulating a range of dosage for use in humans. A dose may be formulated in animal models to achieve a circulating plasma concentration range that includes the IC50, (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical Compositions and Uses Thereof

### Pharmaceutical Compositions

In another aspect, the present invention provides a composition, e.g., a pharmaceutical composition, containing one or a combination of C3b binding molecules (e.g., monoclonal antibodies, or antigen-binding portion(s) thereof), of the present invention, formulated together with a pharmaceutically acceptable carrier. Such compositions may include one or a combination of (e.g., two or more different) binding molecules. For example, a pharmaceutical composition of the invention can comprise a combination of antibodies or agents that bind to different epitopes on the target antigen or that have complementary activities.

Pharmaceutical compositions of the invention also can be administered in combination therapy, i.e., combined with other agents. For example, the combination therapy can include an anti-C3b antibody combined with at least one anti-inflammatory agent. Examples of therapeutic agents that can be used in combination therapy are described in greater detail below in the section on uses of the agents of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for parenteral, administration (e.g., by injection or infusion). As used herein, “parenteral” administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, intraocular (includes intravitreal), subcutaneous, subcuticular, intraarticular, subcapsular, subarachnoid, intraspinal, epidural and intrasternal injection and infusion. Depending on the route of administration, the C3b binding molecule may be coated or provided in a delivery material to protect it from the action of acids and other natural conditions that may inactivate the binding molecule of the present invention.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The C3b binding molecule of the present invention can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of the C3b binding molecule calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such a binding molecule for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months.

Dosage regimens for C3b antibody of the invention include 1 mg/kg body weight or 3 mg/kg body weight by intravenous administration, with the antibody being given using one of the following dosing schedules: every four weeks for six dosages, then every three months; every three weeks; 3 mg/kg body weight once followed by 1 mg/kg body weight every three weeks.

Preferred administration routes of the C3b binding molecules is by topical application to the eye. The ophthalmic compositions are typically administered to the affected eye by applying one to four drops of a sterile solution or suspension, or a comparable amount of an ointment, gel or other solid or semi-solid composition to the surface of the affected eye one to four times a day. The formulations may also be formulated as irrigating solutions that are applied to the affected eye during surgical procedures.

The C3b binding molecule may be formulated in accordance with routine procedures as a pharmaceutical composition adapted for injection intravenously, introperitoneally, or intravitreously. The C3b binding molecule is administered by intravenous injection. A high dose intravenous immunoglobulin (IVIG), as well as F(ab)2-IVIG and even irrelevant human monoclonal antibodies all can bind C3a and C5a and interfere with their function. Basta M. et al. F(ab)′2-mediated neutralization of C3a and C5a anaphylatoxins: a novel effector function of immunoglobulins. Nature Medicine 2003; 9:431-8. A composition comprising the C3b binding molecule may be adapted for intravitreous injection to the eye. Typically, compositions for injection are solutions in sterile isotonic aqueous buffer. Where necessary, the C3b binding molecule may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In one embodiment, suitable doses for the treatment of neovascular (wet) age-related macular degeneration in adult patients is 0.5 milligrams (0.05 milliliters) injected intravitreally into the affected eye once monthly (approximately 28 days). Adequate anesthesia and a broad-spectrum microbicide is given prior to binding molecule injection. Where monthly injections are not feasible, treatment may be reduced to one injection every 3 months after the first 4 injections. In another embodiment, the effective doses of the antibodies for the treatment of neovascular macular degeneration is 0.3 milligrams intravitreally once monthly.

In some methods, two or more binding molecules (e.g., monoclonal antibodies) with different binding specificities are administered simultaneously, in which case the dosage of each antibody administered falls within the ranges indicated. The C3b binding molecule is usually administered on multiple occasions. Intervals between single dosages can be, for example, weekly, monthly, every three months or yearly. Intervals can also be irregular as indicated by measuring blood levels of binding molecule to C3b neo-epitope in the patient. In some methods, dosage is adjusted to achieve a plasma concentration of the C3b binding molecule of about 1-1000 μg/ml and in some methods about 25-300 μg/ml.

Alternatively, a C3b binding molecule can be administered as a sustained release formulation, in which case less frequent administration is required. Dosage and frequency vary depending on the half-life of the C3b binding molecule in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of C3b binding molecule of the invention can result in a decrease in severity of disease symptoms (e.g., a decrease in C3 and/or C5 convertase activity), an increase in frequency and duration of disease symptom-free periods, or a prevention of impairment or disability due to the disease affliction.

A binding molecule of the present invention can be administered by one or more routes of administration using one or more of a variety of methods known in the art. As will be appreciated by the skilled artisan, the route and/or mode of administration will vary depending upon the desired results. Routes of administration for C3b binding molecules of the invention include intravenous, intraocular, intravitreal, intramuscular, intradermal, intraperitoneal, subcutaneous, spinal or other parenteral routes of administration, for example by injection or infusion.

Alternatively, a C3b binding molecule of the invention can be administered by a nonparenteral route, such as a topical, epidermal or mucosal route of administration, for example, intranasally, orally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one aspect, a therapeutic composition of the invention can be administered with a needle-less hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain aspects, the C3b binding molecules of the invention can be formulated to ensure proper distribution in vivo. For example, the blood-brain barrier (“BBB”) or blood retinal barrier (“BRB”) excludes many highly hydrophilic compounds. To ensure that the therapeutic compounds of the invention cross the BBB or BRB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol. 1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### Uses and Methods

The C3b binding molecules described herein have in vitro and in vivo diagnostic and therapeutic utilities. For example, these molecules can be administered to cells in culture, e.g. in vitro or in vivo, or in a subject, e.g., in vivo, to treat, prevent or diagnose a variety of disorders. C3b binding molecules are particularly suitable for treating human patients having, or at risk for, AMD, a condition which in approximately 10% of cases is associated with neovascularization (wet AMD), inflammation and vision loss. C3b binding molecules are also suitable for treating human patients having diseases or disorders such as; nephritis, asthma, reperfusion injury, hemodialysis, rheumatoid arthritis, systemic lupus, psoriasis, multiple sclerosis, transplantation, Alzheimer's disease, aHUS, MPGN II, or any other complement-mediated disease.

When C3b binding molecules are administered together with another agent, the two can be administered sequentially in either order or simultaneously. In some aspects, a C3b binding molecule is administered to a subject who is also receiving therapy with a second agent (e.g., verteporfin). In other aspects, the binding molecule is administered in conjunction with surgical treatments.

Suitable agents for combination treatment with C3b binding molecules include agents known in the art that are able to modulate the activities of complement components (see, e.g., U.S. Pat. No. 5,808,109). Other agents have been reported to diminish complement-mediated activity. Such agents include: amino acids (Takada, Y. et al. Immunology 1978, 34, 509); phosphonate esters (Becker, L. Biochem. Biophy. Acta 1967, 147, 289); polyanionic substances (Conrow, R. B. et al. J. Med. Chem. 1980, 23, 242); sulfonyl fluorides (Hansch, C.; Yoshimoto, M. J. Med. Chem. 1974, 17, 1160, and references cited therein); polynucleotides (DeClercq, P. F. et al. Biochem. Biophys. Res. Commun. 1975, 67, 255); pimaric acids (Glovsky, M. M. et al. J. Immunol. 1969, 102, 1); porphines (Lapidus, M. and Tomasco, J. Immunopharmacol. 1981, 3, 137); several antiinflammatories (Burge, J. J. et al. J. Immunol. 1978, 120, 1625); phenols (Muller-Eberhard, H. J. 1978, in Molecular Basis of Biological Degradative Processes, Berlin, R. D. et al., eds. Academic Press, New York, p. 65); and benzamidines (Vogt, W. et al Immunology 1979, 36, 138). Some of these agents function by general inhibition of proteases and esterases. Others are not specific to any particular intermediate step in the complement pathway, but, rather, inhibit more than one step of complement activation. Examples of the latter compounds include the benzamidines, which block C1, C4 and C5 utilization (see, e.g., Vogt et al. Immunol. 1979, 36, 138).

Additional agents known in the art that can inhibit activity of complement components include K-76, a fungal metabolite from Stachybotrys (Corey et al., J. Amer. Chem. Soc. 104: 5551, 1982). Both K-76 and K-76 COOH have been shown to inhibit complement mainly at the C5 step (Hong et al., J. Immunol. 122: 2418, 1979; Miyazaki et al., Microbiol. Immunol. 24: 1091, 1980), and to prevent the generation of a chemotactic factor from normal human complement (Bumpers et al., Lab. Clinc. Med. 102: 421, 1983). At high concentrations of K-76 or K-76 COOH, some inhibition of the reactions of C2, C3, C6, C7, and C9 with their respective preceding intermediaries is exhibited. K-76 or K-76 COOH has also been reported to inhibit the C3b inactivator system of complement (Hong et al., J. Immunol. 127: 104-108, 1981). Other suitable agents for practicing methods of the present invention include griseofulvin (Weinberg, in Principles of Medicinal Chemistry, 2d Ed., Foye, W. O., ed., Lea & Febiger, Philadelphia, Pa., p. 813, 1981), isopannarin (Djura et al., Aust. J. Chem. 36: 1057, 1983), and metabolites of Siphonodictyon coralli-phagum (Sullivan et al., Tetrahedron 37: 979, 1981).

A combination therapy regimen may be additive, or it may produce synergistic results (e.g., reductions in complement pathway activity more than expected for the combined use of the two agents). In some aspects, combination therapy with a C3b binding molecule and an anti-angiogenic, such as anti-VEGF produces synergistic results (e.g., synergistic reductions in C3b bioactivity).

Also within the scope of the invention are kits consisting of the compositions of the invention and instructions for use. The kit can further contain a least one additional reagent, or one or more additional antibodies of the invention (e.g., an antibody having a complementary activity which binds to an a C3b neo-epitope distinct from the first antibody). Kits typically include a label indicating the intended use of the contents of the kit. The term label includes any writing, or recorded material supplied on or with the kit, or which otherwise accompanies the kit.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated in their entirety by reference.

### EXAMPLES

### Example A

### Generation of Human Antibodies by Phage Display

For the generation of antibodies against C3b, selections with the MorphoSys HuCAL GOLD® phage display library are carried out. HuCAL GOLD® is a Fab library based on the HuCAL® concept in which all six CDRs are diversified, and which employs the CysDisplay™ technology for linking Fab fragments to the phage surface (Knappik et al., 2000 J. Mol. Biol. 296:57-86; Krebs et al., 2001 J. Immunol. Methods 254:67-84; Rauchenberger et al., 2003 J Biol. Chem. 278(40):38194-38205; WO 01/05950, Löhning, 2001).

Phagemid Rescue, Phage Amplification, and Purification

The HuCAL GOLD® library is amplified in 2×YT medium containing 34 μg/ml chloramphenicol and 1% glucose (2×YT-CG). After infection with VCSM13 helper phages at an OD₆₀₀ₙₘ of 0.5 (30 min at 37° C. without shaking; 30 min at 37° C. shaking at 250 rpm), cells are spun down (4120 g; 5 min; 4° C.), resuspended in 2×YT/34 μg/ml chloramphenicol/50 μg/ml kanamycin/0.25 mM IPTG and grown overnight at 22° C. Phages are PEG-precipitated twice from the supernatant, resuspended in PBS/20% glycerol and stored at −80° C.

Phage amplification between two panning rounds is conducted as follows: mid-log phase E. coli TG1 cells are infected with eluted phages and plated onto LB-agar supplemented with 1% of glucose and 34 μg/ml of chloramphenicol (LB-CG plates). After overnight incubation at 30° C., the TG1 colonies are scraped off the agar plates and used to inoculate 2×YT-CG until an OD₆₀₀ₙₘ of 0.5 is reached and VCSM13 helper phages added for infection as described above.

Pannings with HuCAL GOLD®

For the selection of antibodies recognizing C3b neoepitopes, two different panning strategies are applied. In summary, HuCAL GOLD® phage-antibodies are divided into four pools comprising different combinations of VH master genes (pool 1: VH1/5λκ, pool 2: VH3λκ, pool 3: VH2/4/6λκ, pool 4: VH1-6λκ). These pools are individually subjected to three rounds of solid phase panning both on human C3b directly conjugated to sulfolink agarose beads and C3b directly coated to sulfhydrylbind plates. and in addition three of solution pannings on biotinylated C3b antigen.

The first panning variant is solid phase panning against C3b neoepitopes: 2 wells on a sulfhydryl-Bind plate (Corning) are coated with 300 μl of 5 μg/ml C3b—each o/n at 4° C. The coated wells are washed 2× with 350 μl PBS and blocked with 350 μl 5% MPBS for 2 h at RT on a microtiter plate shaker. For each panning about 10¹³ HuCAL GOLD® phage-antibodies are blocked with equal volume of PBST/5% MP for 2 h at room temperature. The coated wells are washed 2× with 350 μl PBS after the blocking. 300 μl of pre-blocked HuCAL GOLD® phage-antibodies are added to each coated well and incubated for 2 h at RT on a shaker. Washing is performed by adding five times 350 μl PBS/0.05% Tween, followed by washing another four times with PBS. Elution of phage from the plate is performed with 300 μl 20 mM DTT in 10 mM Tris/HCl pH8 per well for 10 min. The DTT phage eluate is added to 14 ml of E. coli TG1, which are grown to an OD₆₀₀ of 0.6-0.8 at 37° C. in 2YT medium and incubated in 50 ml plastic tubes for 45 min at 37° C. without shaking for phage infection. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates and phages were rescued and amplified as described above. The second and third rounds of the solid phase panning on directly coated C3b antigen is performed according to the protocol of the first round, but with increased stringency in the washing procedure.

The second panning variant is solution panning against biotinylated human C3b antigen: For the solution panning, using biotinylated C3b antigen coupled to Dynabeads M-280 (Dynal), the following protocol is applied: 1.5 ml Eppendorf tubes are blocked with 1.5 ml 2× Chemiblocker diluted 1:1 with PBS over night at 4° C. 200 μl streptavidin coated magnetic Dynabeads M-280 (Dynal) are washed 1× with 200 μl PBS and resuspended in 200 μl 1× Chemiblocker (diluted in 1×PBS). Blocking of beads is performed in pre-blocked tubes over night at 4° C. Phages diluted in 500 μl PBS for each panning condition are mixed with 500 μl 2× Chemiblocker/0.1% Tween 1 h at RT (rotator). Pre-adsorption of phages is performed twice: 50 μl of blocked Streptavidin magnetic beads are added to the blocked phages and incubated for 30 min at RT on a rotator. After separation of beads via a magnetic device (Dynal MPC-E) the phage supernatant (˜1 ml) is transferred to a new blocked tube and pre-adsorption was repeated on 50 μl blocked beads for 30 min. Then, 200 nM biotinylated C3b is added to blocked phages in a new blocked 1.5 ml tube and incubated for 1 h at RT on a rotator. 100 μl of blocked streptavidin magnetic beads is added to each panning phage pool and incubated 10 min at RT on a rotator. Phages bound to biotinylated C3b are immobilized to the magnetic beads and collected with a magnetic particle separator (Dynal MPC-E). Beads are then washed 7× in PBS/0.05% Tween using a rotator, followed by washing another three times with PBS. Elution of phage from the Dynabeads is performed adding 300 μl 20 mM DTT in 10 mM Tris/HCl pH 8 to each tube for 10 min. Dynabeads are removed by the magnetic particle separator and the supernatant is added to 14 ml of an E. coli TG-1 culture grown to OD₆₀₀ₙₘ of 0.6-0.8. Beads are then washed once with 200 μl PBS and together with additionally removed phages the PBS was added to the 14 ml E. coli TG-1 culture. For phage infection, the culture is incubated in 50 ml plastic tubes for 45 min at 37° C. without shaking. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates, and phages are rescued and amplified as described above.

The second and third rounds of the solution panning on biotinylated C3b antigen are performed according to the protocol of the first round, except with increased stringency in the washing procedure.

Subcloning and Expression of Soluble Fab Fragments

The Fab-encoding inserts of the selected HuCAL GOLD® phagemids are sub-cloned into the expression vector pMORPH®X9_Fab_FH to facilitate rapid and efficient expression of soluble Fabs. For this purpose, the plasmid DNA of the selected clones is digested with XbaI and EcoRI, thereby excising the Fab-encoding insert (ompA-VLCL and phoA-Fd), and cloned into the XbaI/EcoRI -digested expression vector pMORPH®X9_Fab_FH. Fabs expressed from this vector carry two C-terminal tags (FLAG™ and 6×His, respectively) for both, detection and purification.

Microexpression of HuCAL GOLD® Fab Antibodies in E. coli

Chloramphenicol-resistant single colonies obtained after subcloning of the selected Fabs into the pMORPH®X9_Fab_FH expression vector are used to inoculate the wells of a sterile 96-well microtiter plate containing 100 μl 2×YT-CG medium per well and grown overnight at 37° C. 5 μl of each E. coli TG-1 culture is transferred to a fresh, sterile 96-well microtiter plate pre-filled with 100 μl 2×YT medium supplemented with 34 μg/ml chloramphenicol and 0.1% glucose per well. The microtiter plates are incubated at 30° C. shaking at 400 rpm on a microplate shaker until the cultures are slightly turbid (˜2-4 hrs) with an OD₆₀₀ₙₘ of ˜0.5.

To these expression plates, 20 μl 2×YT medium supplemented with 34 μg/ml chloramphenicol and 3 mM IPTG (isopropyl-β-D-thiogalactopyranoside) is added per well (end concentration 0.5 mM IPTG), the microtiter plates are sealed with a gas-permeable tape, and the plates are incubated overnight at 30° C. shaking at 400 rpm.

Generation of whole cell lysates (BEL extracts): To each well of the expression plates, 40 μl BEL buffer (2×BBS/EDTA: 24.7 g/l boric acid, 18.7 g NaCl/l, 1.49 g EDTA/l, pH 8.0) is added containing 2.5 mg/ml lysozyme and incubated for 1 h at 22° C. on a microtiter plate shaker (400 rpm). The BEL extracts are used for binding analysis by ELISA or a BioVeris M-series® 384 analyzer.

Enzyme Linked Immunosorbent Assay (ELISA) Techniques

5 μg/ml of human recombinant C3b antigen in PBS is coated onto 384 well Maxisorp plates (Nunc-immunoplate) o/n at 4° C. After coating, the wells are washed once with PBS/0.05% Tween (PBS-T) and 2× with PBS. Then the wells are blocked with PBS-T with 2% BSA for 2 h at RT. In parallel, 15 μl BEL extract and 15 μl PBS-T with 2% BSA are incubated for 2 h at RT. The blocked Maxisorp plated are washed 3× with PBS-T before 10 μl of the blocked BEL extracts are added to the wells and incubated for 1 h at RT. For detection of the primary Fab antibodies, the following secondary antibodies are applied: alkaline phosphatase (AP)-conjugated AffiniPure F(ab′)₂ fragment, goat anti-human, -anti-mouse or -anti-sheep IgG (Jackson Immuno Research). For the detection of AP-conjugates fluorogenic substrates like AttoPhos (Roche) are used according to the instructions by the manufacturer. Between all incubation steps, the wells of the microtiter plate are washed with PBS-T three times and three times after the final incubation with secondary antibody. Fluorescence can be measured in a TECAN Spectrafluor plate reader.

Expression of HuCAL GOLD® Fab Antibodies in E. coli and Purification

Expression of Fab fragments encoded by pMORPH®X9_Fab_FH in TG-1 cells is carried out in shaker flask cultures using 750 ml of 2×YT medium supplemented with 34 μg/ml chloramphenicol. Cultures are shaken at 30° C. until the OD₆₀₀ₙₘ reaches 0.5. Expression is induced by addition of 0.75 mM IPTG for 20 h at 30° C. Cells are disrupted using lysozyme and Fab fragments isolated by Ni-NTA chromatography (Qiagen, Hilden, Germany). Protein concentrations can be determined by UV-spectrophotometry (Krebs et al. J Immunol Methods 254, 67-84 (2001).

### Example B

### Affinity Maturation of Selected Anti-C3b Neo-Epitope Fabs by Parallel Exchange of LCDR3 and HCDR2 Cassettes

Generation of Fab Libraries for Affinity Maturation

In order to increase the affinity and inhibitory activity of the identified anti-C3b antibodies, Fab clones are subjected to affinity maturation. For this purpose, CDR regions are optimized by cassette mutagenesis using trinucleotide directed mutagenesis (Virnekas et al. Nucleic Acids Res 22, 5600-5607, 1994).

The following paragraph briefly describes a protocol that can be used for cloning of the maturation libraries and Fab optimization. Fab fragments from expression vector pMORPH®X9_Fab_FH are cloned into the phagemid vector pMORPH®25 (U.S. Pat. No. 6,753,136). Two different strategies are applied in parallel to optimize both, the affinity and the efficacy of the parental Fabs.

Phage antibody Fab libraries are generated where the LCDR3 of six selected maturation candidates (“parental” clones) is replaced by a repertoire of individual light chain CDR3 sequences. In parallel, the HCDR2 region of each parental clone is replaced by a repertoire of individual heavy chain CDR2 sequences. Affinity maturation libraries are generated by standard cloning procedures and transformation of the diversified clones into electro-competent E. coli TOP10F′ cells (Invitrogen). Fab-presenting phages are prepared as described in Example 1. Maturation pools corresponding to each library are built and kept separate during the subsequent selection process.

Maturation Panning Strategies

Pannings using the four antibody pools are performed on C3b in solution for three rounds, respectively as described above, solution panning against biotinylated C3b. The selection stringency is increased by reduction of biotinylated antigen from panning round to panning round, by prolonged washing steps and by addition of non-biotinylated antigen for off-rate selection.

Electrochemiluminescene (BioVeris) Based Binding Analysis for Detection of C3b Binding Fab in Bacterial Lysates

Binding of optimized Fab antibodies in E. coli lysates (BEL extracts) to C3b is analyzed in BioVeris M-SERIES® 384 AnalyzerBioVeris, Europe, Witney, Oxforfshire, UK). BEL extracts are diluted in assay buffer (PBS/0.05% Tween20/0.5% BSA) for use in BioVeris screening. Biotinylated C3b is coupled to streptavidin coated paramagnetic beads, Anti-human (Fab)′₂ (Dianova) was ruthenium labeled using the BV-tag™ (BioVeris Europe, Witney, Oxfordshire, UK). This secondary antibody is added to the C3b coupled beads before measuring in the BioVeris M-SERIES® 384 Analyzer. Sequence analysis of hits from the BioVeris screening is conducted to identify Fab clones. Selected Fab antibodies are sub-cloned into IgG1 format.

Determination of Picomolar Affinities Using Solution Equilibrium Titration (SET)

For K_{D} determination, monomer fractions (at least 90% monomer content, analyzed by analytical SEC; Superdex75, Amersham Pharmacia) of Fab are used. Electrochemiluminescence (ECL) based affinity determination in solution and data evaluation can be performed essentially as described by Haenel et al., 2005. A constant amount of Fab is equilibrated with different concentrations (serial 3ⁿ dilutions) of C3b in solution. Biotinylated C3b coupled to paramagnetic beads (M-280 Streptavidin, Dynal), and BV-tag™ (BioVeris Europe, Witney, Oxfordshire, UK) labeled anti-human (Fab)′₂ (Dianova) is added and the mixture incubated for 30 min. Subsequently, the concentration of unbound Fab is quantified via ECL detection using the M-SERIES® 384 analyzer (BioVeris Europe).

Affinity determination to C3b of another species (e.g., chimpanzee or cynomolgus) in solution is done essentially as described above, replacing the human C3b with the chimpanzee or cynomolgus C3b. For detection of free Fab, biotinylated C3b coupled to paramagnetic beads is used. Affinities are calculated according to Haenel et al. (2005 Anal Biochem 339, 182-184).

### Example C

### Detection of Complement Proteins by Hemolysis Assay

Specimens of aqueous humor and vitreous are obtained from patients with age-related macular degeneration. The patients undergo surgery for the underlying disease, and specimens are obtained at the start of intraocular surgery. Samples (100-200 μl of aqueous humor and 200 to 300 μl of vitreous) are obtained undiluted and used immediately or stored at −80° C.

Aqueous humor and vitreous samples are obtained from normal human patients and incubated with normal human serum at 37° C. for 2 hours. The mixture is assayed for inhibition of the classical and alternative complement pathways using standard CH50 and AH50 hemolytic assays. In these assays normal human serum is obtained from normal healthy subjects and used as the source of complement and are stored in aliquots at −80° C. Normal human serum is also treated with fractions obtained after microcentrifugation and gel filtration column as conventionally used in the art. Total complement activity in aqueous and vitreous is also determined.

CH50 Assay

The CH50 assay is described Kabat, E. A. et al Experimental Immunochemistry 1961. pp. 133-239. Normal human serum is used as the source of complement and is stored in aliquots at −80° C. Total complement activity in aqueous and vitreous alone was also determined and utilizes sheep erythrocytes (SRBC) as target cells (red blood cells from other species can be used, e.g., chicken red blood cells). A suspension containing SRBC/ml is prepared in the GVB²⁺ buffer (gelatin/Veronal-buffered saline with Ca²⁺ and Mg²⁺), pH 7.35. Hemolysin (rabbit anti-sheep antiserum) is titrated to determine the optimal dilution to sensitize SRBC. Diluted hemolysin mixed with an equal volume of SRBC and the whole is incubated at 37° C. for 15 minutes. This results in antibody-coated erythrocytes (EA). EA are incubated with serial twofold dilutions of the normal human serum or similar dilution of the mixture of normal human serum and the test sample at 37° C. for 1 hour. Test sample is defined as unfractionated aqueous/vitreous, filtrate, and retain obtained after microconcentration obtained after size exclusion column. Normal human serum incubated with GVB²⁺ buffer is used as the control. Background control is obtained by incubating EA with buffer alone (serum was not added), and total lysis (100% hemolysis) is determined by adding distilled water to EA. The reaction is stopped using 1.2 ml of ice-cold 0.15 M NaCl, the mixture is spun to pellet the unlysed cells, and the optical density of the supernatant is determined spectrophotometrically (412 nm). The percentage of hemolysis is determined relative to the 100% lysis control.

Complement activity is quantitated by determining the serum dilution required to lyse 50% of cells in the assay mixture. The results are expressed as the reciprocal of this dilution in CH₅₀ units/ml of serum.

AH50 Assay

An AH₅₀ assay is carried out using the standard methods described in the Kabat, et al which depend on lysis of unsensitized rabbit erythrocytes (Erab) by human serum by activation of the alternative pathway. Activation of the calcium-dependent classical pathway is prevented by addition of the calcium chelator ethylene glycol tetraacetic acid (EGTA) to the assay buffer, and magnesium, necessary for both pathways, is added to the buffer. A cell suspension of rabbit RBC is prepared in the GVB-Mg²⁺-EGTA buffer. A serial 1.5-fold dilution of normal human serum or similar dilution of the mixture of normal human serum and the test sample is prepared in GVB-Mg²⁺-EGTA buffer, and 100 μl of each serum dilution is added to 50 μl of standardized Erab. Normal human serum incubated with GVB-Mg²⁺-EGTA buffer is used as the control. The mixture is then incubated at 60 minutes at 37° C. in a shaking water bath to keep cells in suspension, and 1.2 ml of ice-cold NaCl (0.15 M) is used to stop the reaction. The tubes are spun at 1250 g, at 4° C., for 10 minutes to pellet the cells, and the optical density of the supernatant is determined spectrophotometrically (412 nm). In the total lysis control tube 100 μl of distilled water is added to 50 μl Erab suspension, and the percentage of hemolysis is determined relative to 100% lysis control. Complement activity is quantitated by determining the serum dilution required to lyse 50% of cells in the assay mixture. The results are expressed as the reciprocal of this dilution in AH50 units/ml of serum.

What is claimed is:

1. An isolated binding molecule comprising an antigen binding portion that binds to a C3b neo-epitope.

2. An isolated C3b binding molecule comprising an antigen binding portion that specifically binds to a C3b epitope, wherein the antigen binding portion binds to an epitope of human C3b within or overlapping one of the following:

| | |
|---|---|
| (a) amino acids GEDTVQSLTQG of; | SEQ ID NO: 1 |
| (b) amino acids DEDIIAEENIVSRSEF of; | SEQ ID NO: 2 |
| (c) amino acids IRMNKTVAVRT of; | SEQ ID NO: 3 |
| (d) amino acids SDQVPDTESET of; | SEQ ID NO: 4 |
| (e) amino acids VAQMTED of; | SEQ ID NO: 5 |
| (f) amino acids FVKRAP of; | SEQ ID NO: 6 |
| (g) amino acids KDKNRWEDPGKQLYN of; | SEQ ID NO: 7 |
| (h) amino acids CTRYRGDQDATMS; | SEQ ID NO: 8 |
| or | |
| (i) amino acids GFAPDTDDLKQLANGV. | SEQ ID NO: 9 |

3. The C3b binding molecule of claim 1, wherein the antigen binding portion is cross reactive with a C3b antigen of a non-human primate.

4. The C3b binding molecule of claim 1, wherein the antigen binding portion is cross reactive with a C3b antigen of a rodent species.

5. The C3b binding molecule of claim 1, wherein the antigen binding portion binds to a linear epitope.

6. The C3b binding molecule of claim 1, wherein the antigen binding portion binds to a non-linear epitope.

7. The C3b binding molecule of claim 1, wherein the antigen binding portion binds to a human C3b antigen with a K_{D} equal to or less than 1 nM.

8. The C3b binding molecule of claim 1, wherein the antigen binding portion binds to C3b antigen of a non-human primate with a K_{D} equal to or less than 5 nM.

9. The C3b binding molecule of claim 1, wherein the antigen binding portion is an antigen binding portion of a human antibody.

10. The C3b binding molecule of claim 1, wherein the antibody is a human or humanized antibody.

11. The C3b binding molecule of claim 1, wherein the antigen binding portion is an antigen binding portion of a monoclonal antibody.

12. The C3b binding molecule of claim 1, wherein the antigen binding portion is an antigen binding portion of a polyclonal antibody.

13. The C3b binding molecule of claim 1, wherein the C3b binding molecule is a chimeric antibody.

14. The C3b binding molecule of claim 1, wherein the C3b binding molecule comprises an Fab fragment, an Fab′ fragment, an F(ab′)₂, or an Fv fragment of the antibody.

15. The C3b binding molecule of claim 1, wherein the C3b binding molecule comprises a single chain Fv.

16. The C3b binding molecule of claim 1, wherein the C3b binding molecule comprises a diabody.

17. The C3b binding molecule of claim 1, wherein the antigen binding portion is derived from an antibody of one of the following isotypes: IgG1, IgG2, IgG3 or IgG4.

18. The C3b binding molecule of claim 1, wherein the antigen binding portion is derived from an antibody of one of the following isotypes: IgG1, IgG2, IgG3 or IgG4 in which the Fc sequence has been altered relative to the normal sequence in order to modulate effector functions or alter binding to Fc receptors.

19. The C3b binding molecule of claim 18 wherein the Fc sequence has been altered at amino acid residues 234 or 235.

20. The C3b binding molecule of claim 1, wherein the C3b binding molecule inhibits MAC production in a cell.

21. The C3b binding molecule of claim 1, wherein the C3b binding molecule inhibits C3b binding to a convertase.

22. The C3b binding molecule of claim 21 wherein the C3b binding molecule inhibits C3 binding to the C3 or C5 convertase.

23. The C3b binding molecule of claim 1, wherein the C3b binding molecule inhibits proteolytic activity of C3 or C5 convertases.

24. The C3b binding molecule of claim 1, wherein the C3b binding molecule, when contacted with a cell or properdin under conditions in which C3b antigen is present, reduces the generation of: (i) C3 or C5 convertase; or (ii) C5a or MAC; or (iii) C3a or iC3b or C3b on the cell or surface, relative to inhibition in the absence of the C3b binding molecule.

25. A pharmaceutical composition comprising the C3b binding molecule of claim 1.

26. A method of inhibiting MAC synthesis in (cell), the method comprising contacting a cell or properdin with a C3b binding molecule.

27. A peptide consisting of an amino acid sequence at least 90% identical to an amino acid selected from Table 1.

28. A method of modulating C3b activity in a subject, the method comprising administering to the subject a C3b binding molecule that modulates a cellular activities mediated by the complement system.

29. A method of treating an ocular disorder in a subject in need thereof comprising administering to the subject an effective amount of a composition of claim 25.

30. The method of claim 28, wherein the subject's level of MAC is reduced by at least 5%, relative to the level of MAC in a subject prior to administering the composition.

31. The method of claim 28, wherein the subject is also receiving therapy with a second agent.

32. The method of claim 28, wherein the subject has, or is at risk for, AMD.

33. The method of claim 32, wherein the subject exhibits the dry form of AMD or is at risk for the wet form of AMD.


SEQUENCE LISTING (37 sequences)

SEQ ID NO: 1 - protein, 11 aa, Homo Sapien
GEDTVQSLTQG

SEQ ID NO: 2 - protein, 16 aa, Homo Sapien
DEDIIAEENIVSRSEF

SEQ ID NO: 3 - protein, 11 aa, Homo Sapien
IRMNKTVAVRT

SEQ ID NO: 4 - protein, 11 aa, Homo Sapien
SDQVPDTESET

SEQ ID NO: 5 - protein, 7 aa, Homo Sapien
VAQMTED

SEQ ID NO: 6 - protein, 6 aa, Homo Sapien
FVKRAP

SEQ ID NO: 7 - protein, 15 aa, Homo Sapien
KDKNRWEDPGKQLYN

SEQ ID NO: 8 - protein, 13 aa, Homo Sapien
CTRYRGDQDATMS

SEQ ID NO: 9 - protein, 16 aa, Homo Sapien
GFAPDTDDLKQLANGV

SEQ ID NO: 10 - protein, 12 aa, Homo Sapien
DSLSSQNQLGVL

SEQ ID NO: 11 - protein, 14 aa, Homo Sapien
PIEDGSGEVVLSRK

SEQ ID NO: 12 - protein, 12 aa, Homo Sapien
GVQNPRAEDLVG

SEQ ID NO: 13 - protein, 7 aa, Homo Sapien
DGSPAYR

SEQ ID NO: 14 - protein, 9 aa, Homo Sapien
QGEDTVQSL

SEQ ID NO: 15 - protein, 8 aa, Homo Sapien
KQELSEAE

SEQ ID NO: 16 - protein, 12 aa, Homo Sapien
VREPGQDLVVLP

SEQ ID NO: 17 - protein, 15 aa, Homo Sapien
VVKSGQSEDRQPVPG

SEQ ID NO: 18 - protein, 10 aa, Homo Sapien
VEDLKEPPKN

SEQ ID NO: 19 - protein, 12 aa, Homo Sapien
YNYRQNQELKVR

SEQ ID NO: 20 - protein, 10 aa, Homo Sapien
ATTKRRHQQT

SEQ ID NO: 21 - protein, 12 aa, Homo Sapien
HFISDGVRKSLK

SEQ ID NO: 22 - protein, 11 aa, Homo Sapien
SDQVPDTESET

SEQ ID NO: 23 - protein, 9 aa, Homo Sapien
TPSGCGEQN

SEQ ID NO: 24 - protein, 8 aa, Homo Sapien
ELIKKGYT

SEQ ID NO: 25 - protein, 12 aa, Homo Sapien
EKQKPDGVFQED

SEQ ID NO: 26 - protein, 9 aa, Homo Sapien
LRNNNEKDM

SEQ ID NO: 27 - protein, 15 aa, Homo Sapien
TTAKDKNRWEDPGKQ

SEQ ID NO: 28 - protein, 13 aa, Homo Sapien
CTRYRGDQDATMS

SEQ ID NO: 29 - protein, 16 aa, Homo Sapien
GFAPDTDDLKQLANGV

SEQ ID NO: 30 - protein, 10 aa, Homo Sapien
HSEDDCLAFK

SEQ ID NO: 31 - protein, 12 aa, Homo Sapien
SGSDEVQVGQQR

SEQ ID NO: 32 - protein, 12 aa, Homo Sapien
LSSDFWGEKPNL

SEQ ID NO: 33 - protein, 15 aa, Homo Sapien
EDECQDEENQKQCQD

SEQ ID NO: 34 - protein, 9 aa, Homo Sapien
NREFKSEKG

SEQ ID NO: 35 - protein, 3 aa, Homo Sapien
QNL

SEQ ID NO: 36 - protein, 9 aa, Homo Sapien
ETEKRPQDA

SEQ ID NO: 37 - protein, 2 aa, Homo Sapien
SD